(19) United States
(12) Patent Application Publication
(10) Pub. No.: US 2022/0152231 A1
(43) Pub. Date: May 19, 2022

(54) PROCESS OF PREPARING POLYMERIC NANOPARTICLES THAT CHELATE RADIOACTIVE ISOTOPES AND HAVE A SURFACE MODIFIED WITH SPECIFIC MOLECULES TARGETING THE PSMA RECEPTOR AND THEIR USE

(71) Applicant: NANOTHEA S.A., Warszawa (PL)

(72) Inventors: Tomasz CIACH, Warszawa (PL); Magdalena JANCZEWSKA, Warszawa (PL); Grzegorz PIKUS, Warszawa (PL); Konstancia KOPYRA, Warszawa (PL)

(21) Appl. No.: 17/440,902

(22) PCT Filed: Mar. 19, 2019
(86) PCT No.: PCT/IB2019/052218

§ 371 Date: Sep. 20, 2021

(51) Int. Cl. A61K 51/12; A61K 51/04; A61K 51/06; C08L 5/02

(52) U.S. Cl. CPC A61K 51/1244; A61K 51/0497; B82Y 5/00; C08L 5/02; A61K 51/065

(57) ABSTRACT

Process for preparation of polymeric nanoparticles that chelate radioactive isotopes and have their surface modified with specific molecules targeting PSMA receptor on the surface of cancer cells, with a targeting agent modified by a linker molecule attaching to free aldehyde groups present in the dextran chain. Polymeric nanoparticles that chelate radioactive isotopes synthesized according to the claimed process for use in therapy and diagnostics of prostate cancer and metastatic cancer cells as well as other affected cells for which the nanoparticles show the affinity.

### TECHNICAL FIELD

The subject of the invention is a process for the preparation of polymer nanoparticles capable of lasting and stable chelating of radioisotopes, with attached targeting agent for the PSMA receptor present on the surface of neoplastic cells. The described particles are used mostly for therapy and diagnostics of prostate cancer cells, metastatic prostate cancer cells and focal therapy (targeted brachytherapy).

### BACKGROUND ART

According to the data of the American Cancer Society, approx. 14.1 million cases of cancer and about 8.2 million of deaths from cancer were recorded worldwide in 2012. In 2015, 1,658,370 new cancer cases were forecast to appear in the USA, with 220,800 representing prostate cancer. 589,430 of those cases (35.5%) are forecast to end with death, with 27,540 of them to be caused by prostate cancer. Estimates indicate that in 2030 there will be approximately 21.7 million new cases of cancer, of which about 13 million will end in death. The above values arise from the positive birth rate and the increasingly strong and common ageing of the population. Those forecasts may keep growing, due to the civilisation- and lifestyle-related determinants (smoking, bad diet, lack of physical activity).

Prostate cancer diagnostics is well-defined. Currently used hybrid methods of ultrasound imaging and MRI permit increasingly definitive identification of sites significantly affected within the prostate. Thanks to this the subsequent, still irreplaceable, biopsy more precise. However, what remains a challenge for modem medicine is the therapy of metastatic cells. The currently known solutions using radioisotopes can be divided into three sub-groups: (i) conjugates guided by targeting molecules with chelated radioisotope (Prostascint®), (ii) small molecules using metabolic changes as a targeting element (Axumin®) or (iii) free mixtures of radioisotopes (Xofigo®) using natural accumulation of radioisotopes in bone tissue, i.e. in the most frequent site of metastatic prostate cancer cells.

Conjugates are compounds consisting of three components: a chelator (usually a bifunctional chelator), a linker and a targeting molecule (aptamer, oligopeptide, antibody, antimetabolite).

Antimetabolites and small molecules (glucose) are absorbed and used by neoplasms to a greater extent. This mechanism of action permits universal targeting for various types of cancers. Compounds of this group are used in such markers as FDG (fluorine-18 labelled glucose) and Axumin® (fluorine-18 labelled fluciclovine) or C-choline (carbon-11 choline). A characteristic feature shared by the listed products is a radioisotope that is an integral part of a carbon compound skeleton. This, however, entails a need for “hot” synthesis and rapid transport of the radiopharmaceutical.

Due to the natural biological affinity of radioisotopes to bone cells and their tendency to accumulate in the bone tissue, there are radiopharmaceuticals available in the market which are administered to patients in the form of a solution of unbound radioisotopes. The application of such preparations is justified mostly in the therapy of patients with metastatic prostate cancer. Xofigo® from Bayer may be an example of such preparations. Administering a free isotope means that the activity of the radiation is non-specific. It affects both the prostate metastatic cells located in bone tissue, as well as bone-forming and bone-resorbing cells indispensable for proper functioning of the bone skeleton.

Nanoparticle-based therapeutics are a beneficial solution, since a single agent may supply the drug and the contrast medium for prostate cancer through the recognition of surface receptors highly expressed by the cancer cells. Prostate-specific membrane antigen (PSMA) is a type II transmembrane glycoprotein detected for the first time in the prostate cancer human cell line LNCaP. According to the available knowledge, the membrane of prostate cancer cells has over ten times more PSMA receptors than healthy prostate gland cells [The Prostate 2004, 58, 200-210.]

PSMA expression usually increases as the prostate cancer progresses and metastases, providing a perfect target for effective cancer cell targeting along with imaging and cancer treatment, especially in the case of more aggressive forms of the disease. Over the past two decades, a large number of low-molecule PSMA inhibitors have been tested, such as phosphonates, phosphates and phosphoamidates, as well as thiols and urea. Furthermore, high PSMA levels were identified in the endothelial cells of cancers associated with systems of other solid tumours, including breast, lungs, colon and pancreas.

Targeted therapy in cancer treatment is an area that is gaining momentum both in pre-clinical and in clinical trials. Specific delivery of drugs to cancer cells using nanoparticles may take place either through extracellular release of therapeutics from the nanoparticles to the tumour microenvironment (passive transport) or through intracellular drug release by way of endocytosis (active transport).

It seems highly beneficial to use an active targeted therapy that involves attaching another substance to the drug nanoparticle, the affinity of such substance for the membrane receptors of cancer cells being exceptionally high, which significantly increases the binding of the drug with the cancer cell and the uptake of the drug (Moghimi et al. 2001). This makes it important to find the right ligand that would match the receptor characteristic of a particular cancer type.

### PURPOSE OF THE INVENTION

The object of the invention is to provide specifically targeted polymeric nanoparticles carrying radioisotopes to prostate cancer cells, prostate cancer metastatic cells and any cancers where overexpression of the PSMA receptor has been confirmed.

The object of the invention is to provide a process for the preparation of nanoparticles with a surface modified with specific molecules targeting the PSMA receptor. Another object of the invention is to provide specifically targeted nanoparticles that may be used for therapy (focal brachytherapy) and for PET, PET/MR diagnostics.

### SUMMARY OF THE INVENTION

The subject of the invention is the process for preparing polymeric nanoparticles that chelate radioactive isotopes and have their surface modified with specific molecules targeting the PSMA receptor on the surface of cancer cells. The invention also covers nanoparticles obtained according to the claimed method and their use.

The process for preparing polymeric nanoparticles that chelate radioactive isotopes and have their surface modified with specific molecules targeting the PSMA receptor on the surface of cancer cells comprises several stages, in which:

a) a dextran chain is oxidised to polyaldehyde by means of periodate.

b) a targeting agent modified by a linker molecule is attached to free aldehyde groups present in the dextran chain,

c) a folding agent in the form of hydrophobic or hydrophilic amine, diamine or polyamine is attached, with one or two amino groups of the folding agent attaching to aldehyde groups,

d) the resulting imine bonds are reduced to amine bonds,

e) to the free amino group of the attached folding agent, a chelator molecule is attached via an amide bond,

f) the resulting mixture is purified,

g) the nanoparticle fraction is subjected to lyophilisation.

Preferably, the mixture from stage (f) is purified through dialysis.

Preferably, the cells where the PSMA receptor is present are prostate cancer cells and prostate cancer metastatic cells.

Also preferably, the cells where the PSMA receptor is present are breast, lung, colon and pancreatic cancer cells.

According to the process of the invention, the level of aldehyde group substitution with the targeting agent is from 1 to 50%, preferably from 2.5 to 5%.

As chelators, derivatives of DOTA, DTPA and/or NOTA are used.

As the targeting agent, α,α-urea of glutamic acid and lysine is used.

As the linker, preferably 2,5-dioxopyrrolidin-1-yl 2,2-dimethyl-4-oxo-3,8,11,14,17,20-hexaoxa-5-azatricos-23-ate (PEGs) is used.

As the folding agent hydrophobic or hydrophilic amines, diamines, or polyamines are used, such as dodecylamines, diaminooctanes, diaminodecanes (DAD), polyether diamines, polypropylene diamines and block copolymer diamines.

According to the process of the invention, the resulting nanoparticles are labelled radiochemically. Preferably, the nanoparticles are labelled with isotopes in which the decay pathway includes beta plus decay, beta minus decay, gamma emitter, such as Cu-64, Ga-68, Ga-67, It-90, In-111, Lu-177, Ak-227, and Gd (for MR).

The invention also includes polymeric nanoparticles chelating radioactive isotopes, with a surface modified by specific molecules targeting the PSMA receptor as obtained according to the above process, for use in diagnostics and therapy.

The invention includes the use of the polymeric nanoparticles chelating radioactive isotopes in diagnostics with the use of Positron Emission Tomography (PET), hybrid Positron Emission Tomography/Magnetic Resonance (PET/MRI).

The invention also covers the use of the polymeric nanoparticles chelating radioactive isotopes in focal brachytherapy.

Furthermore, the invention includes the use of the polymeric nanoparticles chelating radioactive isotopes in the therapy and diagnostics of prostate cancer and prostate cancer metastatic cells and the remaining affected cells for which the nanoparticles display the affinity.

The nanoparticles of the invention may be obtained with the use of such polymers as dextran, hyaluronic acid, cellulose and its derivatives. Polymers are used both in the native form and after being oxidised to aldehyde groups or carboxyl groups. The synthesis of nanoparticles is carried out by the formation of imines and their subsequent reduction and esters of carboxylic groups.

As folding agents, hydrophobic or hydrophilic amines, diamines, polyethylene glycols, polypropylene glycols or short block-block polymers are used, in which one or two amine groups can undergo the reaction.

As the targeting agent, α,α-urea of glutamic acid and lysine, i.e. Glu-CO-Lys (GuL) is used, with the following formula

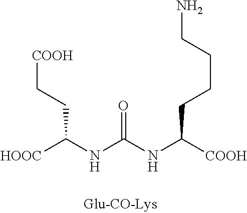 [SMILES: NCCCC[C@H](NC(=O)N[C@@H](CCC(=O)O)C(=O)O)C(=O)O]

This small-molecule compound that is a urea derivative of two amino acids has a high affinity for the PSMA receptor. It forms hydrogen bonds with amino acids and coordinate bonds with the zinc atom in the active centre inside the protein. As a result, it binds strongly to the receptor, forming a complex that penetrates the cells by way of endocytosis. GuL is a compound that can be selectively modified in the primary amino group, which opens considerable possibilities for the bioconjugation of that particle.

The linker molecule to which the targeting molecule (GuL) is attached was selected and applied because of the structure of the receptor protein. Used as the linker are w-amino acid derivatives, including oligopeptide derivatives, where the amino group is protected by such groups as tert-butyloxycarbonyl group (Boc), 9-fluorenylmethylcarbonyl group (Fmoc), benzyloxycarbonyl group (Cbz), benzyl group (Bn), triphenylmethyl group (Tr), while the carbonyl group occurs as free acid (carboxyl group) or as an ester. The overall structural formula of the linker used is presented in the figure below,

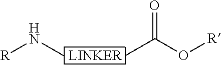

where R and R′ may have the structure of:

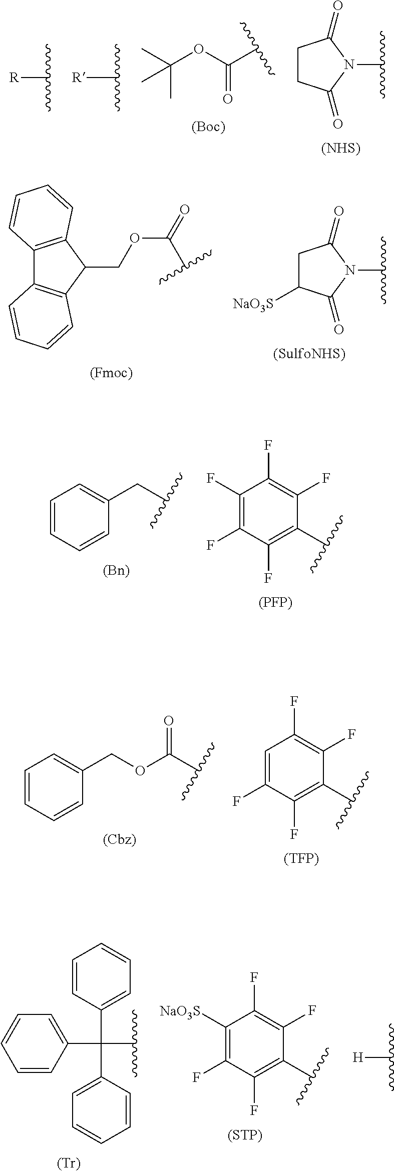

Due to the protein structure of the receptor to which the targeting agent shows affinity, the following types of linkers are used:

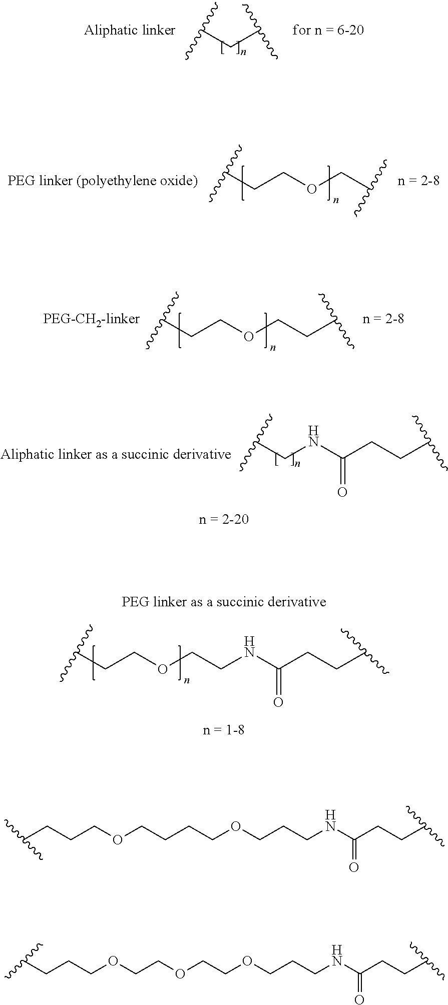

It is particularly preferred to use a linker containing polyethylene oxide (PEG) where n is 5 (PEGs) or n is 4 (PEG₄), as presented below:

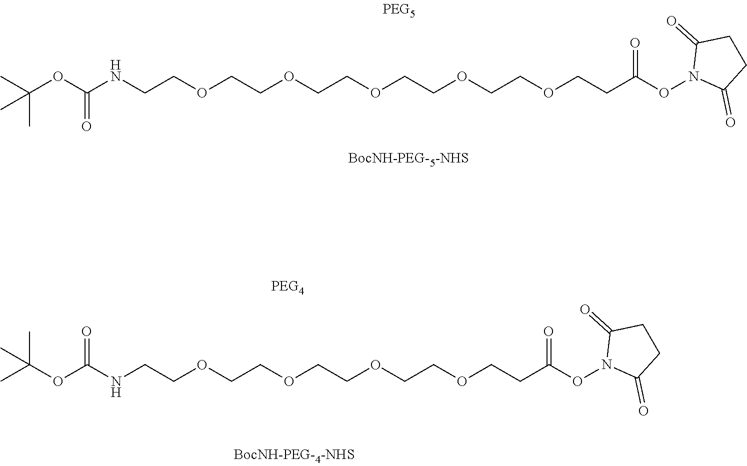 [SMILES: CC(C)(C)OC(=O)NCCOCCOCCOCCOCCOCCC(=O)ON1C(=O)CCC1=O ; CC(C)(C)OC(=O)NCCOCCOCCOCCOCCC(=O)ON1C(=O)CCC1=O]

The nanoparticles of the invention are obtained through chemical modification of the polymer chain, followed by formation of a dynamic micelle structure through self-organisation in an aqueous environment.

At the initial stage, the dextran chain is oxidised to polyaldehyde dextran (PAD).

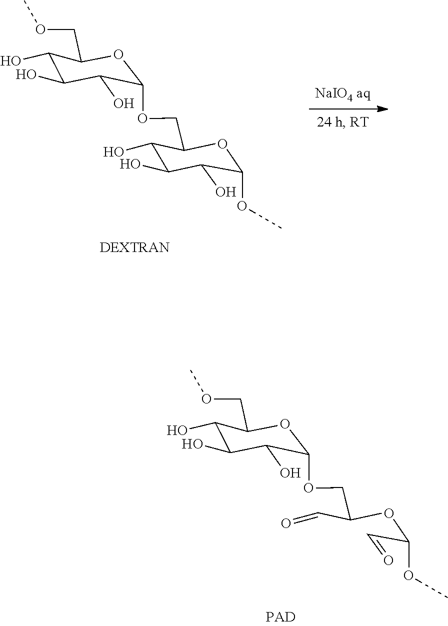

Dextran is oxidised using periodate to form aldehyde groups. Aldehyde groups are formed without the polymer chain being broken.

The determination of the aldehyde groups formed in the oxidation process is necessary for proper calculation of the quantities of the targeting agent and folding agent to be added. The formulations are prepared with the preservation of the percentage proportions, to ensure process repeatability and similarity between subsequent series of prepared nanoparticles. The number of aldehyde groups is 200 to 800 μmol/1 g of PAD, preferably 300 to 600 μmol/1 g of PAD.

Before linking the targeting agent to the nanoparticle, the targeting agent is combined with the linker. Used in the reaction in the form of triesters, Glu-CO-Lys (GuL) undergoes modification through cross-linking with the linker to extend its amine branch. This stage of the process will provide the inhibitor—the targeting molecule with the precise access to the pocket of the PSMA receptor active site. At the same time the inhibitor, after being combined with the nanoparticle, will be adequately exposed on its surface.

The next stage involves attaching, to the aldehyde groups of polyaldehyde dextran (PAD), the previously prepared targeting agent (GuL) already attached to the linker, where the imination reaction leads to the formation of the Schiff base. Afterwards, the folding agent in the form of a lipophilic diamine is attached to the PAD aldehyde groups, which results in the formation of further imine bonds.

The imine bonds formed are reduced using a borohydride ethanol solution. It may be a sodium or a potassium borohydride or cyanoborohydride. Subsequently, the chelator molecules are attached to the free amine group coming from the diamine attached to the dextran chain. The chelator molecule is attached through the conjugation of amine with the NHS ester (N-hydroxysuccinimide ester) of the chelator molecule.

The crucial stage of preparing a product ready for labelling is the purification of the formulation through dialysis.

Dialysis is carried out for water or a proper buffer for 12-72 h, preferably 24-48 h, with frequent fluid exchange. The volumetric ratio of the external fluid to the sample being purified is 20:1 to 200:1, preferably 100:1. After the chelator molecule is attached, the post-reaction mixture is purified against an acetic buffer with pH of 5.0, and after the folic acid (FA) molecule is attached, the mixture is purified against phosphate buffer with pH of 7.4.

The purified nanoparticles are then subjected to lyophilisation, which makes it possible to store them in the form of dry foam for at least 3 months. After being re-combined with water, the nanoparticles reorganise within approx. 20 minutes, gently stirred in the target buffer.

The final nanoparticle preparation stage may involve radiochemical labelling.

The nanoparticles according to the invention are labelled with isotopes in which decay pathway includes beta plus decay, beta minus decay, gamma emitter decay. Those are such isotopes as Cu-64, Ga-68, Ga-67, It-90, In-111, Lu-177, Ak-227 and Gd (for the MRI). This makes the invention useful for both therapeutic and diagnostic purposes. Diagnostics may use various available methods: PET, SCEPT, MRI and their hybrids, e.g. PET/MRI.

The use of such prepared nanoparticles in imaging diagnostics increases the chance of completely curing patients suffering from prostate cancer or from metastatic prostate cancer due to early cancer detection and simultaneous targeted therapy, with a possibility of monitoring the progress of treatment.

### BRIEF DESCRIPTION OF DRAWINGS

The figures enclosed to the description which illustrate the invention present what follows:

FIG. 1—fluorescence assay of the PSMA receptor enzyme activity inhibition for nanoparticles with aldehyde groups substituted with the GuL targeting agent in 10% (BCS 0277), 30% (BCS 0290) and 2.5% BCS 0319) and without the substitution (Control without nanoparticles) for various concentrations of nanoparticle solutions used in the analysis, i.e. 16 μg, 4 μg, 1.6 μg, 0.4 μg, 0.16 μg.

FIG. 2—fluorescence assay of the PSMA inhibition by nanoparticles with GuL without the linker (408) and with the linker (277) for various quantities of the targeting agent, i.e. 8000 ng, 800 ng, 80 ng and 8 ng.

The object of the invention is illustrated in the preferred embodiments described below.

### EXAMPLE 1

Preparation of Nanoparticles with 10% Substitution of Aldehyde Groups with the GuL Targeting Agent at 90% Substitution with the DAD Folding Agent (BCS277)

1.1. Oxidation of Dextran to Polyaldehyde Dextran (PAD)

Dextran Oxidation Reaction:

5.00 g of dextran was dissolved in 100 ml ultrapure water. 0.66 g of sodium periodate was added. The oxidation reaction was continued overnight in the dark at room temperature. The product was purified through dialysis for 72 hours in one hundred-fold volume of the ultrapure water, with the water changed at least twice. The water was removed by evaporation at 40° C.

Determination of Aldehyde Groups in PAD:

100 μl of 0.8 mM hydroxylamine hydrochloride solution, 300 μl of 0.6 M acetate buffer with pH 5.8 and 20-100 μl of PAD was added to a 2 ml tube, and then ultrapure water (0-80 μl) was added up to a total volume 500 μl. The assay was conducted for three different PAD volumes (20, 60 and 100 μl). A control sample was prepared: 100 μl of 0.8 mM hydroxylamine hydrochloride solution, 300 μl of 0.6 M acetate buffer with pH 5.8 and 100 μl of ultrapure water was added to a tube. The samples were mixed, incubated at 95° C. for 15 minutes, and then incubated at room temperature for 5 minutes. 500 μl of 0.05% TNBS solution was added to every sample. The samples were mixed, incubated in the dark at room temperature for 60 minutes. Once the incubation was completed, the sample absorbance was measured at the wavelength of 500 nm. 300 μl of 0.6 M of acetate buffer with pH 5.8 mixed with 200 μl of ultrapure water was used as a blank sample. On the basis of these determinations, the content of aldehyde groups of 480.3 μmol/1 g PAD was determined.

1.2. Reaction of Glu-CO-Lys(OBu^{t})₃NH₂ with the Linker PEGs

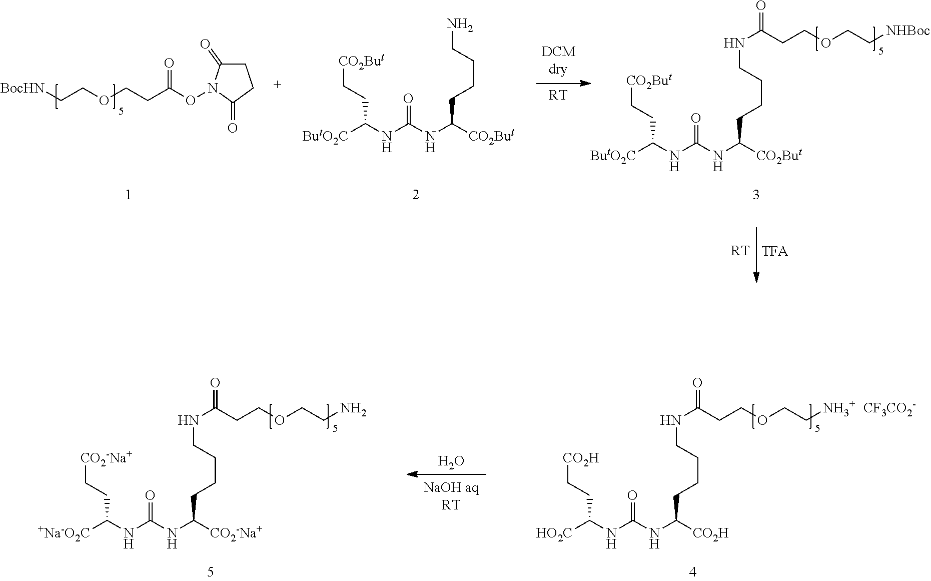

10.40 mg (0.0205 mmol) of the linker (compound 1) was dissolved in 0.5 ml of anhydrous methylene chloride. Afterwards, 10.00 mg (0.0205 mmol) of α,α-urea of glutamic acid and lysine in the form of tert-butyl triesters (compound 2) and 4 μl of DIPEA were added. The reaction was carried out for 24 h at room temperature. After that time, 150 μl of TFA was added, and stirring was continued over the next 24 h at room temperature. The solvent was evaporated, the oily residue was dissolved in 0.5 ml of ultrapure water, and then alkalised with a 5M sodium hydroxide solution to pH>11 against a universal indicator paper. Thus prepared aqueous solution of linker-modified GuL (compound 5) was used for the next stage of the synthesis without purification.

1.3. Formation of Dextran Nanoparticles with Attached Targeting Agent Glu-CO-Lys.

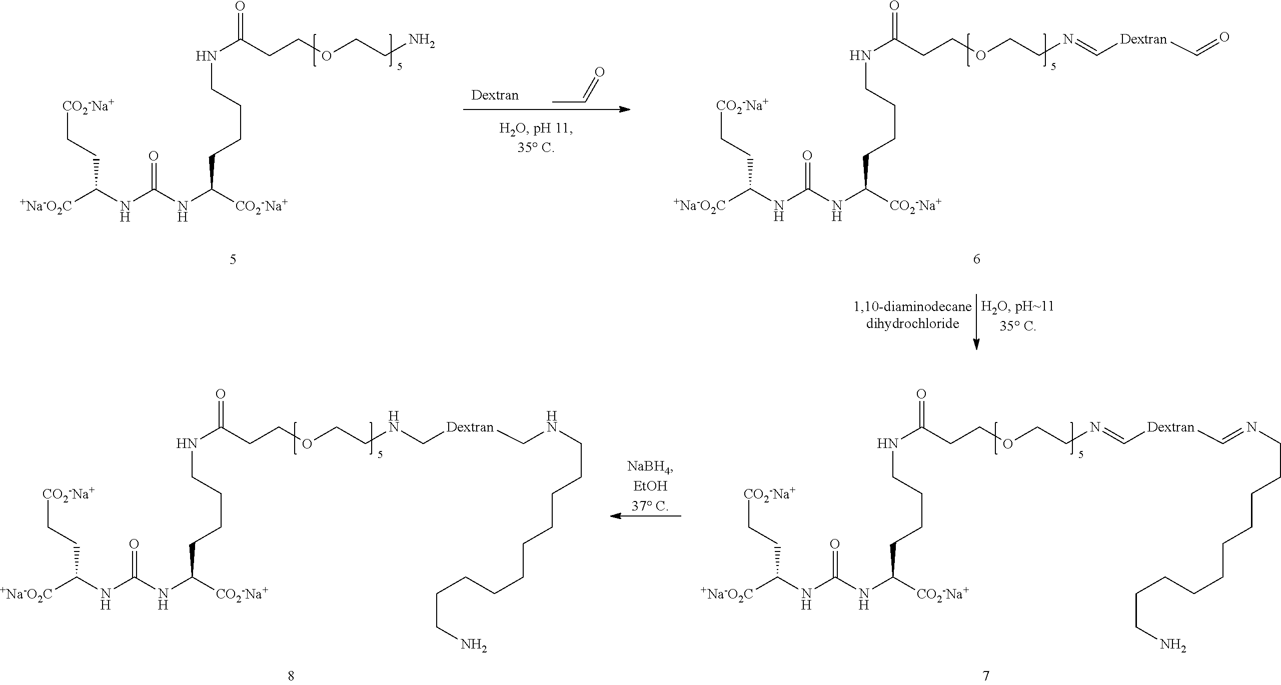

427 mg of PAD (containing 205.1 μmol CHO) was dissolved in 4.3 ml of ultrapure water to give a 10% (w/v) solution. The aqueous solution of linker-modified Glu-CO-Lys (compound 5) was added to this mixture. In thus prepared reaction mixture, a 0.5M NaOH solution was used to bring the pH to 11.00, and the mixture was stirred at 30° C. for 60 minutes, resulting in modified polyaldehyde dextran (compound 6). After this time, 2.27 ml of a 2% (w/v) ultrapure water solution of 1,10-diaminodecane dihydrochloride was added, and the reaction mixture thus obtained was stirred at 30° C. for 10 minutes, with pH controlled and adjusted to 10 every 20 minutes. After the end of the reaction, a 0.5M HCl solution was used to bring the pH to 7.4. Afterwards, 1.60 ml of a 1% (w/v) ethanol solution of sodium borohydride was added. The reduction reaction was carried out at 37° C. for 60 minutes. After the end of the reaction, the pH was brought to 7.4 with a 0.5M HCl solution. The final product 8 was purified by dialysis in one hundred-fold volume of the ultrapure water for 48 h, with water changed six times. Water was removed from thus purified nanoparticles by lyophilisation.

1.4. DOTA Chelator Attachment to Nanoparticles Containing the GuL Targeting Agent

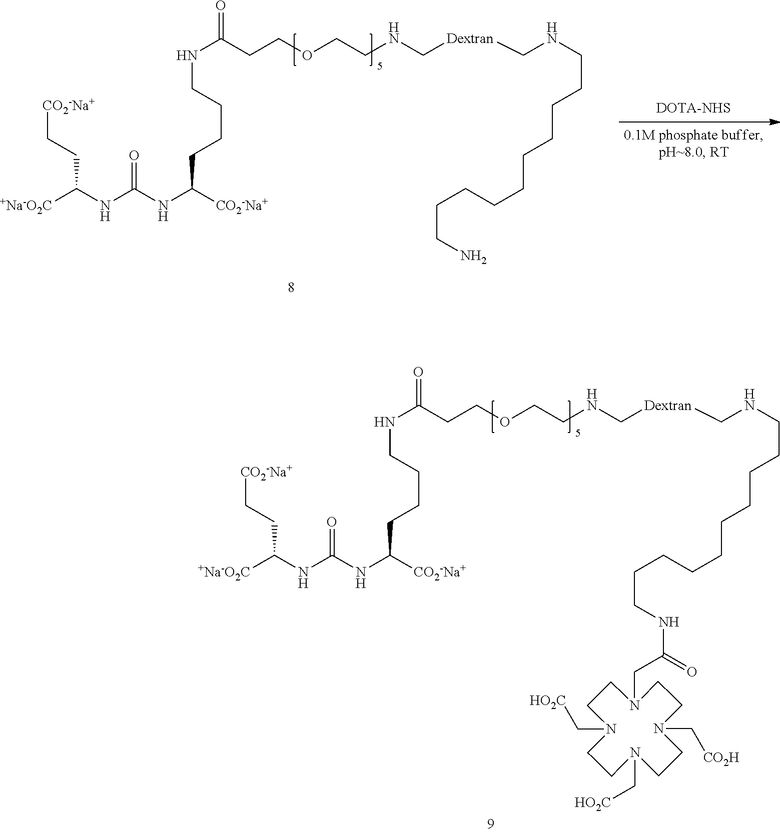

100 mg of nanoparticles lyophilisate (compound 8) was dissolved in 2.0 ml of 0.1M phosphate buffer of pH 8.0. Afterwards, 0.5 ml of DOTA-NHS suspension in ultrapure water, containing 18.5 mg of the chelator, was added. Thus prepared reaction mixture was stirred at room temperature for 90 minutes. The product was purified by dialysis against one hundred-fold volume of 10 mM acetate buffer solution with pH of 5.0 for 48 hours, with the buffer solution changed six times. Water was removed from thus purified nanoparticles (compound 9) by lyophilisation.

### EXAMPLE 2

Preparation of Nanoparticles with 30% Substitution of Aldehyde Groups with the GuL Targeting Agent at 70% Substitution with the DAD Folding Agent (BCS290)

2.1. Oxidation of Dextran to Polyaldehyde Dextran (PAD)

Dextran Oxidation Reaction:

5.00 g of dextran was dissolved in 100 ml ultrapure water. 0.66 g sodium periodate was added. The oxidation reaction was continued overnight in the dark at room temperature. The product was purified through dialysis for 72 hours in one hundred-fold volume of ultrapure water, with the water changed at least twice. The water was removed by evaporation at 40° C.

Determination of Aldehyde Groups in PAD:

100 μl of 0.8 mM hydroxylamine hydrochloride solution, 300 μl of 0.6 M acetate buffer with pH of 5.8 and 20-100 μl of PAD were added to a 2 ml tube, and then ultrapure water (0-80 μl) was added up to a total volume of 500 μl. The assay was conducted for three different PAD volumes (20, 60 and 100 μl). A control sample was prepared: 100 μl of 0.8 mM hydroxylamine hydrochloride solution, 300 μl of 0.6 M acetate buffer with pH of 5.8 and 100 μl of ultrapure water were added to a tube. The samples were mixed, incubated at 95° C. for 15 minutes, and then incubated at room temperature for 5 minutes. 500 μl of 0.05% TNBS solution was added to every sample. The samples were mixed, incubated in the dark at room temperature for 60 minutes. Once the incubation was completed, the sample absorbance was measured at wavelength of 50) nm. 300 μl of 0.6 M acetate buffer of pH 5.8 mixed with 200 μl of ultrapure water was used as a blank sample. Such assays determined a content of aldehyde groups of 508.1 μmol/1 g PAD.

2.2. Reaction of Glu-CO-Lys(OBu^{t})₃NH₂ with the Linker PEGs.

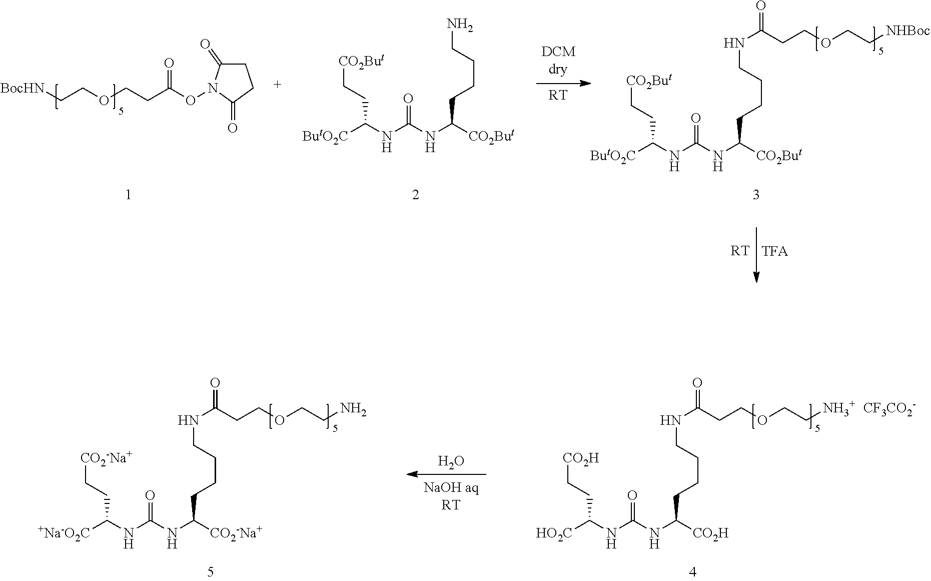

15.50 mg (0.0307 mmol) of the linker (compound 1) was dissolved in 0.75 ml of anhydrous methylene chloride. Afterwards, 15.00 mg (0.0307 mmol) α,α-urea of glutamic acid and lysine in the form of tert-butyl triesters (compound 2) and 6 μl of DIPEA were added. The reaction was carried out for 24 h at room temperature. After that time, 234 μl of TFA was added, and stirring was continued over the next 24 h at room temperature. The solvent was evaporated, the oily residue was dissolved in 0.75 ml of ultrapure water, and then alkalised using 5M sodium hydroxide solution to pH>11 against a universal indicator paper. Thus prepared aqueous solution of linker-modified GuL (compound 5) was used for the next stage of the synthesis without purification.

2.3. Formation of Dextran Nanoparticles with Attached Targeting Agent GuL.

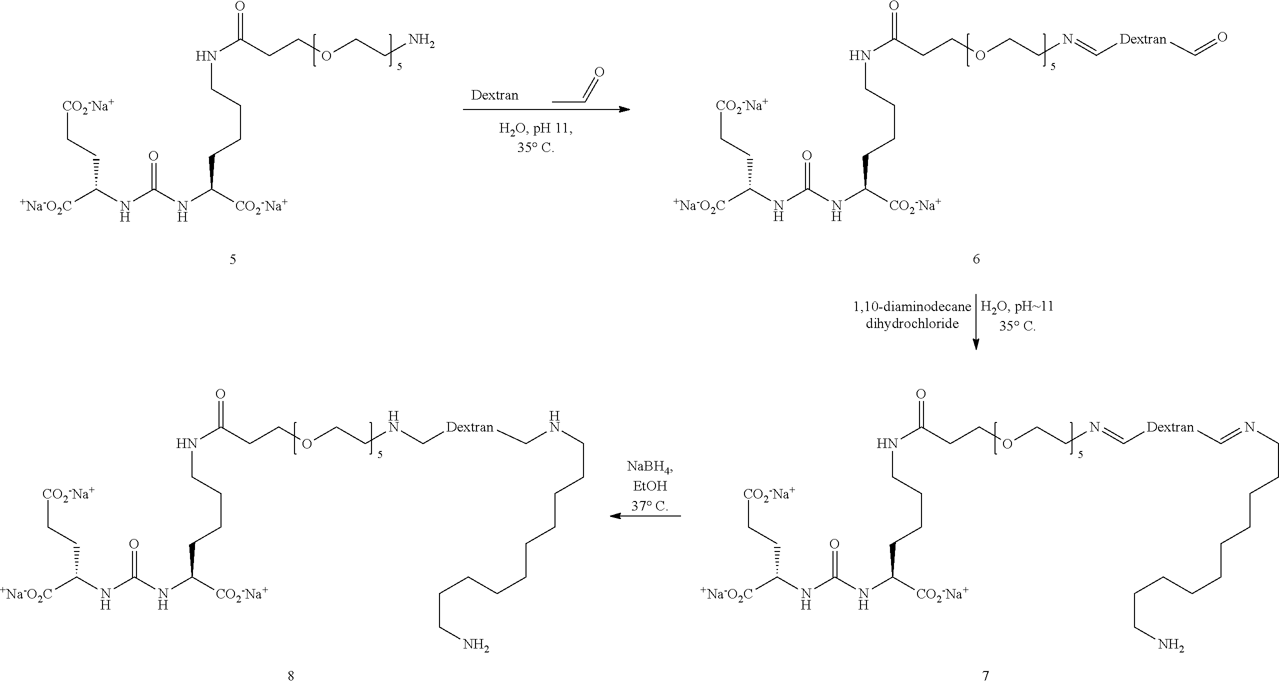

200 mg of PAD (containing 101.6 μmol CHO) was dissolved in 2.0 ml of ultrapure water to give a 10% (w/v) solution. The aqueous solution of linker-modified GuL (compound 5) was added to that mixture. In thus prepared reaction mixture, a 0.5M NaOH solution was used to bring the pH to 11.00, and the mixture was stirred at 30° C. for 60 minutes, resulting in modified polyaldehyde dextran (compound 6). After this time, 0.87 ml of 2% (w/v) ultrapure water solution of 1,10-diaminodecane dihydrochloride was added, and thus obtained reaction mixture was stirred at 30° C. for 10 minutes, with pH controlled and adjusted to 10 every 20 minutes. After the end of the reaction, 0.5M HCl solution was used to bring the pH to 7.4. Afterwards, 0.88 ml of 1% (w/v) ethanol solution of sodium borohydride was added. The reduction reaction was carried out at 37° C. for 60 minutes. After the end of the reaction, the pH was brought to 7.4 using 0.5M HCl solution. The final product 8 was purified by dialysis in one hundred-fold volume of the ultrapure water for 48 h, with water changed six times. Water was removed from thus purified nanoparticles by lyophilisation.

2.4. DOTA Chelator Attachment to Nanoparticles Containing the GuL Targeting Agent

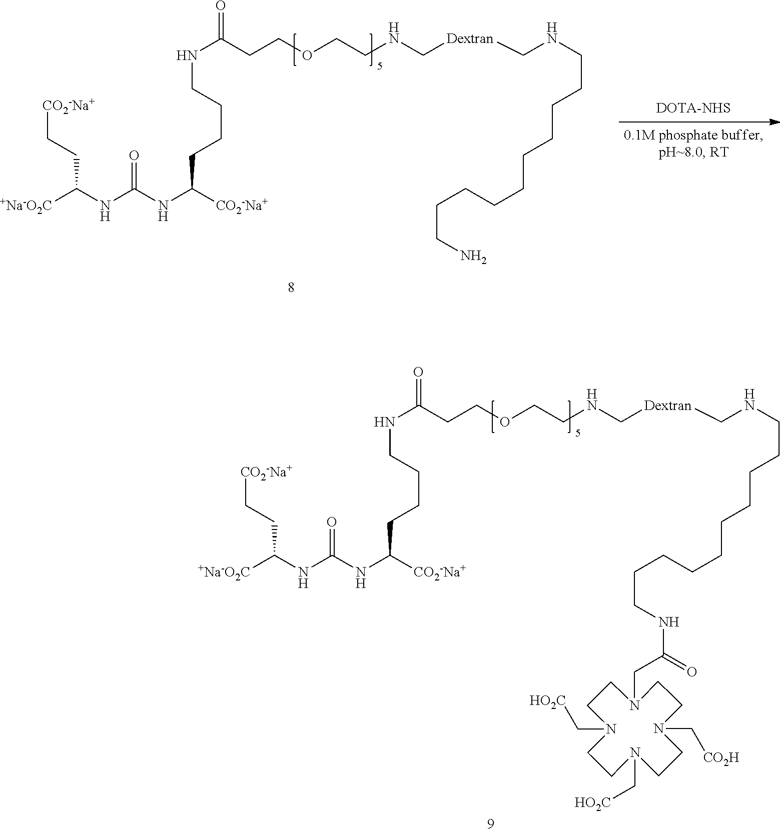

100 mg of nanoparticles lyophilisate (compound 8) was dissolved in 2.0 ml of 0.1M phosphate buffer of pH 8.0. Afterwards, 0.5 ml of DOTA-NHS suspension in ultrapure water, containing 18.5 mg of chelator was added. Thus prepared reaction mixture was stirred at room temperature for 90 minutes. The product was purified through dialysis against one hundred-fold volume of 10 mM acetate buffer with pH of 5.0 for 48 hours, with the buffer solution changed six times. Water was removed from thus purified nanoparticles (compound 9) by lyophilisation.

### EXAMPLE 3

Obtaining Nanoparticles with 5% Aldehyde Group Substitution with the GuL Targeting Agent at 95% Substitution with the DAD Folding Agent (BCS 318)

3.1. Oxidation of Dextran to Polyaldehyde Dextran (PAD)

Dextran Oxidation Reaction:

5.00 g of dextran was dissolved in 100 ml ultrapure water. 0.66 g sodium periodate was added. The oxidation reaction was continued overnight in the dark at room temperature. The product was purified through dialysis for 72 hours in one hundred-fold volume of ultrapure water, with the water changed at least twice. The water was removed by evaporation at 40° C.

Determination of Aldehyde Groups in PAD:

100 μl of 0.8 mM hydroxylamine hydrochloride solution, 300 μl of 0.6 M acetate buffer with pH of 5.8 and 20-100 μl of PAD were added to a 2 ml tube, and then ultrapure water (0-80 μl) was added up to total volume 500 μl. The assay was conducted for three different PAD volumes (20, 60 and 100 μl). A control sample was prepared: 100 μl of 0.8 mM hydroxylamine hydrochloride solution, 300 μl of 0.6 M acetate buffer with pH of 5.8 and 100 μl of ultrapure water were added to a tube. The samples were mixed, incubated at 95° C. for 15 minutes, and then incubated at room temperature for 5 minutes. 500 μl of a 0.05% TNBS solution was added to every sample. The samples were mixed, incubated in the dark at room temperature for 60 minutes. Once the incubation was completed, the sample absorbance was measured at the wavelength of 500 nm. 300 μl of 0.6 M of acetate buffer with pH 5.8 mixed with 200 μl of ultrapure water was used as a blank sample. Such assays determined a content of aldehyde groups of 480.3 μmol/1 g PAD.

3.2. Reaction of Glu-CO-Lys(OBu^{t})₃NH₂ with the Linker PEG₅.

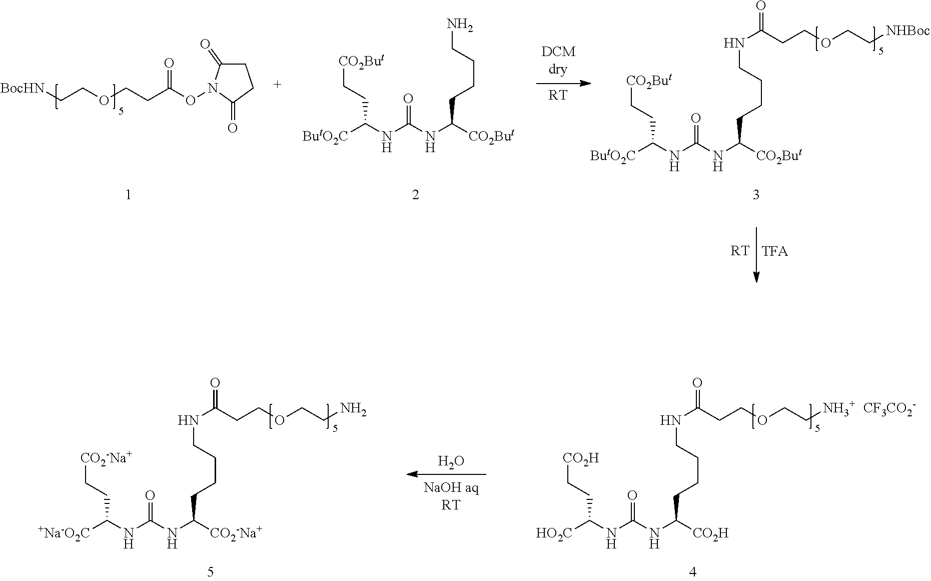

10.40 mg (0.0205 mmol) of the linker (compound 1) was dissolved in 0.5 ml of anhydrous methylene chloride. Afterwards, 10.00 mg (0.0205 mmol) of α,α-urea of glutamic acid and lysine in the form of tert-butyl triesters (compound 2) and 4 μl of DIPEA were added. The reaction was carried out for 24 h at room temperature. After that time, 150 μl of TFA was added, and the mixing was continued over the next 24 h at room temperature. The solvent was evaporated, the oily residue was dissolved in 0.5 ml of ultrapure water, and then alkalised using 5M sodium hydroxide solution to pH>11 against a universal indicator paper. Thus prepared aqueous solution of linker-modified GuL (compound 5) was used for the next stage of the synthesis without purification.

3.3. Formation of Dextran Nanoparticles with Attached Targeting Agent Glu-CO-Lys.

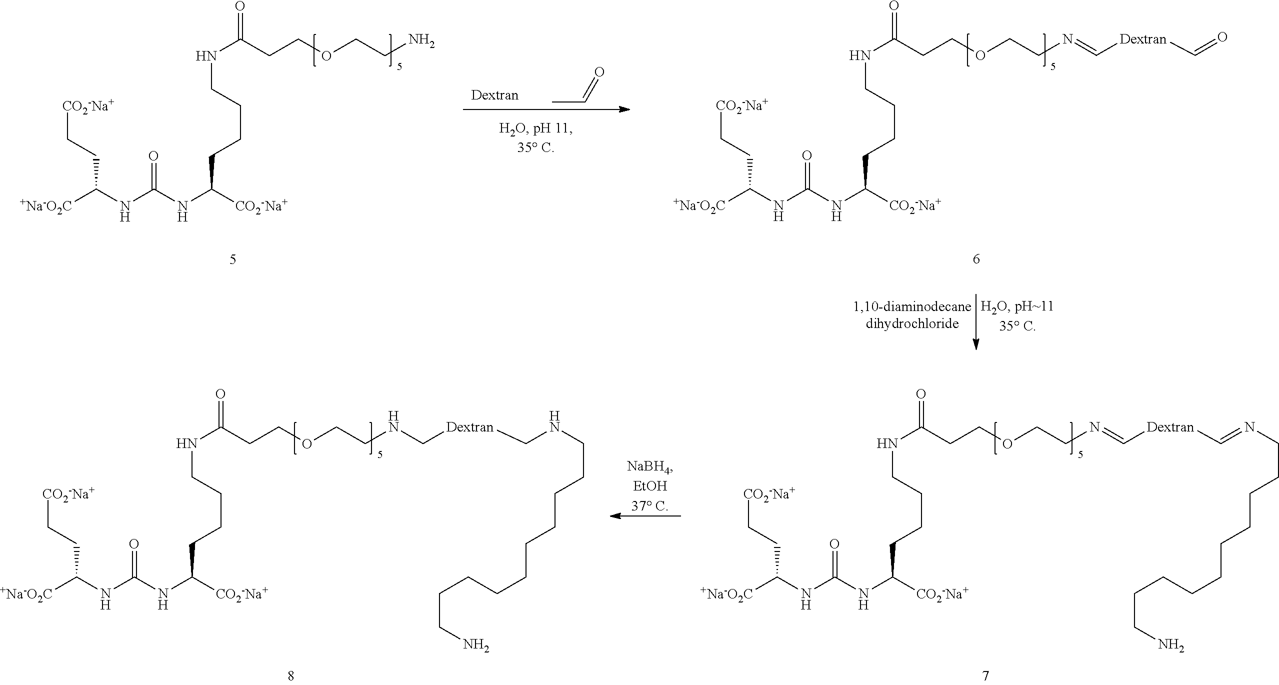

854 mg of PAD (comprising 410.2 μmol CHO) was dissolved in 8.54 ml of ultrapure water to obtain a 10% (w/v) solution. The aqueous solution of linker-modified GuL (compound 5) was added to that mixture. In thus prepared reaction mixture, 0,5M NaOH solution was used to establish pH of 11.00, and the mixture was stirred at 30° C. for 60 minutes, resulting in modified polyaldehyde dextran (compound 6). After that time, 4.78 ml of 2% (w/v) ultrapure water solution of 1,10-diaminodecane dihydrochloride was added, and thus obtained reaction mixture was stirred at 30° C. for 10 minutes, with pH controlled and adjusted to 10 every 20 minutes. After the end of the reaction, 0.5M HCl solution was used to bring the pH to 7.4. Afterwards, 3.18 ml of 1% (w/v) ethanol solution of sodium borohydride was added. The reduction reaction was carried out at 37° C. for 60 minutes. After the end of the reaction, the pH was brought to 7.4 with 0.5M HCl solution. The final product 8 was purified by dialysis in one hundred-fold volume of ultrapure water for 48 h, with water changed six times. Water was removed from thus purified nanoparticles by lyophilisation.

3.4. DOTA Chelator Attachment to Nanoparticles Containing the GuL Targeting Agent

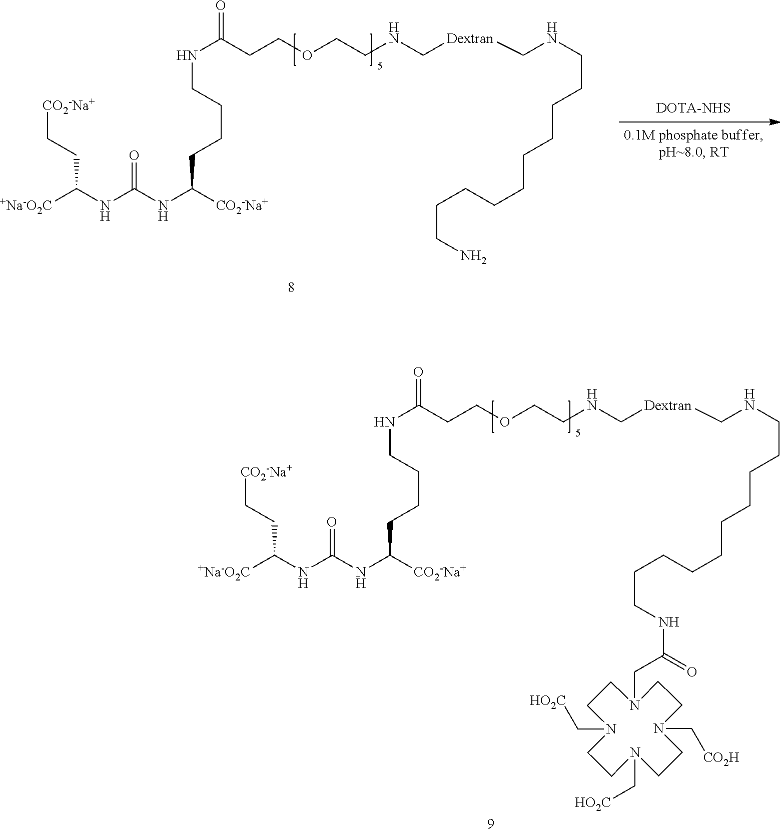

100 mg of nanoparticles lyophilisate (compound 8) was dissolved in 2.0 ml of 0.1M phosphate buffer of 8.0. Afterwards, 0.5 ml of DOTA-NHS suspension in ultrapure water, containing 18.5 mg of the chelator, was added. Thus prepared reaction mixture was stirred at room temperature for 90 minutes. The product was purified through dialysis against one hundred-fold volume of 10 mM acetate buffer with pH of 5.0 for 48 hours, with the buffer solution changed six times. Water was removed from thus purified nanoparticles (compound 9) by lyophilisation.

### EXAMPLE 4

Obtaining Nanoparticles with 2.5% Aldehyde Group Substitution with the GuL Targeting Agent at 97.5% Substitution with the DAD Folding Agent (BCS 319)

4.1. Oxidation of Dextran to Polyaldehyde Dextran (PAD)

Dextran Oxidation Reaction:

5.00 g of dextran was dissolved in 100 ml ultrapure water. 0.66 g sodium periodate was added. The oxidation reaction was continued overnight in the dark at room temperature. The product was purified through dialysis for 72 hours in one hundred-fold volume of ultrapure water, with the water changed at least twice. The water was removed by evaporation at 40° C.

Determination of Aldehyde Groups in PAD:

100 μl of 0.8 mM hydroxylamine hydrochloride solution, 300 μl of 0.6 M acetate buffer with pH of 5.8 and 20-100 μl of PAD were added to a 2 ml tube, and then ultrapure water (0-80 μl) was added up to a total volume 500 μl. The assay was conducted for three different PAD volumes (20, 60 and 100 μl). A control sample was prepared: 100 μl of 0.8 mM hydroxylamine hydrochloride solution, 300 μl of 0.6 M acetate buffer with pH of 5.8 and 100 μl of ultrapure water were added to a tube. The samples were mixed, incubated at 95° C. for 15 minutes, and then incubated at room temperature for 5 minutes. 500 μl of 0.05% TNBS solution was added to every sample. The samples were mixed, incubated in the dark at room temperature for 60 minutes. Once the incubation was completed, the sample absorbance was measured at wavelength of 500 nm. 300 μl of 0.6 M of acetate buffer with pH 5.8 mixed with 200 μl of ultrapure water was used as the blank sample. Such assays determined a content of aldehyde groups of 480.3 μmol/1 g PAD.

4.2. Reaction of Glu-CO-Lys(OBu^{t})₃NH₂ with the Linker PEGs.

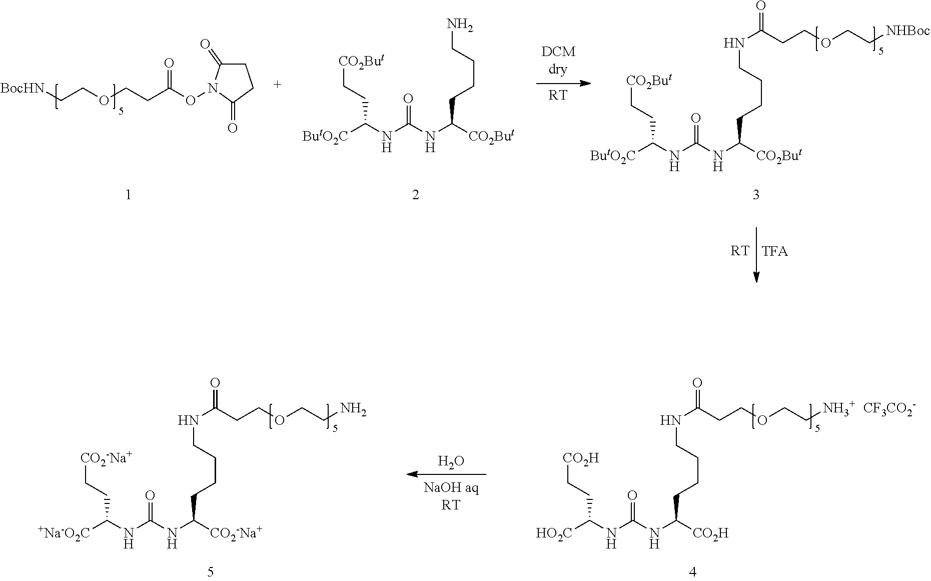

5.20 mg (0.01025 mmol) of the linker (compound 1) was dissolved in 0.25 ml of anhydrous methylene chloride. Afterwards, 5.00 mg (0.01025 mmol) of α,α-urea of glutamic acid and lysine in the form of tert-butyl triesters (compound 2) and 2 μl of DIPEA were added. The reaction was carried out for 24 h at room temperature. After that time, 75 μl of TFA was added, and the mixing was continued over the next 24 h at room temperature. The solvent was evaporated, the oily residue was dissolved in 0.25 ml of ultrapure water and then alkalised using 5M sodium hydroxide solution to pH>11 against a universal indicator paper. Thus prepared aqueous solution of linker-modified GuL (compound 5) was used for the next stage of the synthesis without purification.

4.3. Formation of Dextran Nanoparticles with Attached Targeting Agent Glu-CO-Lys.

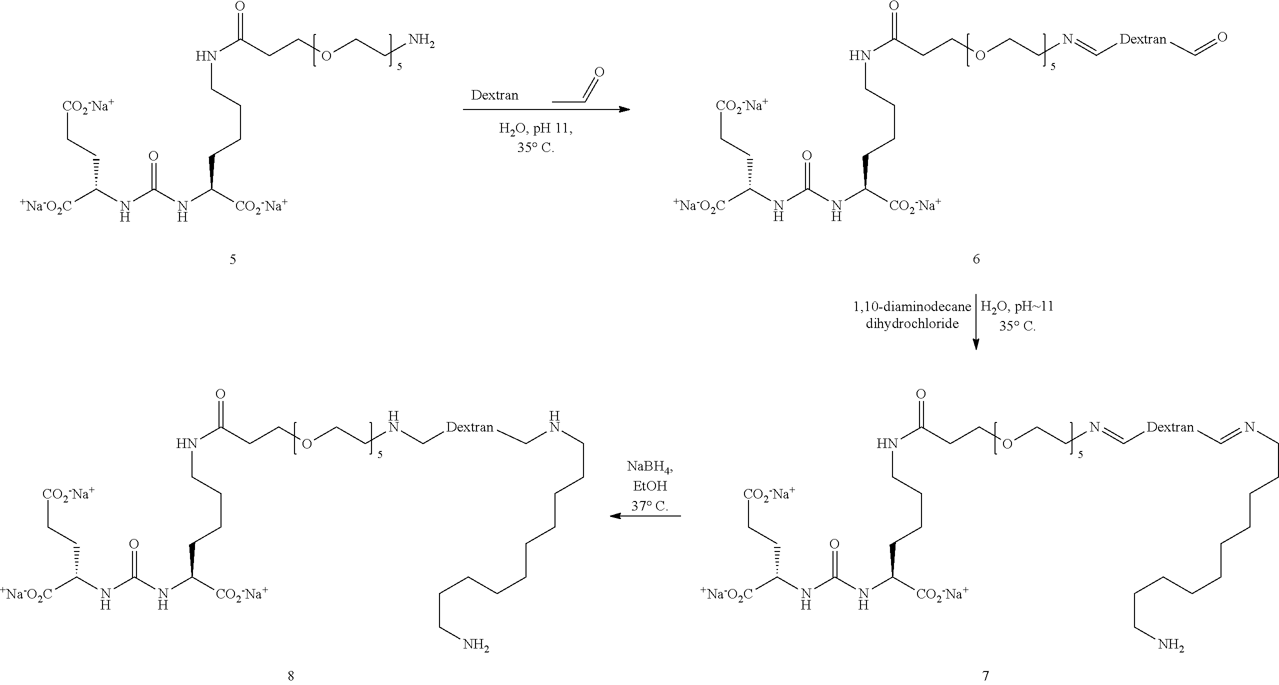

854 mg of PAD (containing 410.2 μmol CHO) was dissolved in 8.54 ml of ultrapure water to obtain a 10% (w/v) solution. The aqueous solution of linker-modified GuL (compound 5) was added to that mixture. In such prepared reaction mixture, 0.5M NaOH solution was used to establish pH of 11.00, and the mixture was stirred at 30° C. for 60 minutes, resulting in modified polyaldehyde dextran (compound 6). After that time, 4.90 ml of 2% (w/v) ultrapure water solution of 1,10-diaminodecane dihydrochloride was added, and thus obtained reaction mixture was stirred at 30° C. for 10 minutes, with pH controlled and adjusted to 10 every 20 minutes. After the end of the reaction, a 0.5M HCl solution was used to bring the pH to 7.4. Afterwards, 3.14 ml of 1% (w/v) ethanol solution of sodium borohydride was added. The reduction reaction was carried out at 37° C. for 60 minutes. After the end of the reaction, the pH was brought to 7.4 using 0.5M HCl solution. The final product 8 was purified by dialysis in one hundred-fold volume of the ultrapure water for 48 h, with water changed six times. Water was removed from thus purified nanoparticles by lyophilisation.

4.4. DOTA Chelator Attachment to Nanoparticles Containing the GuL Targeting Agent

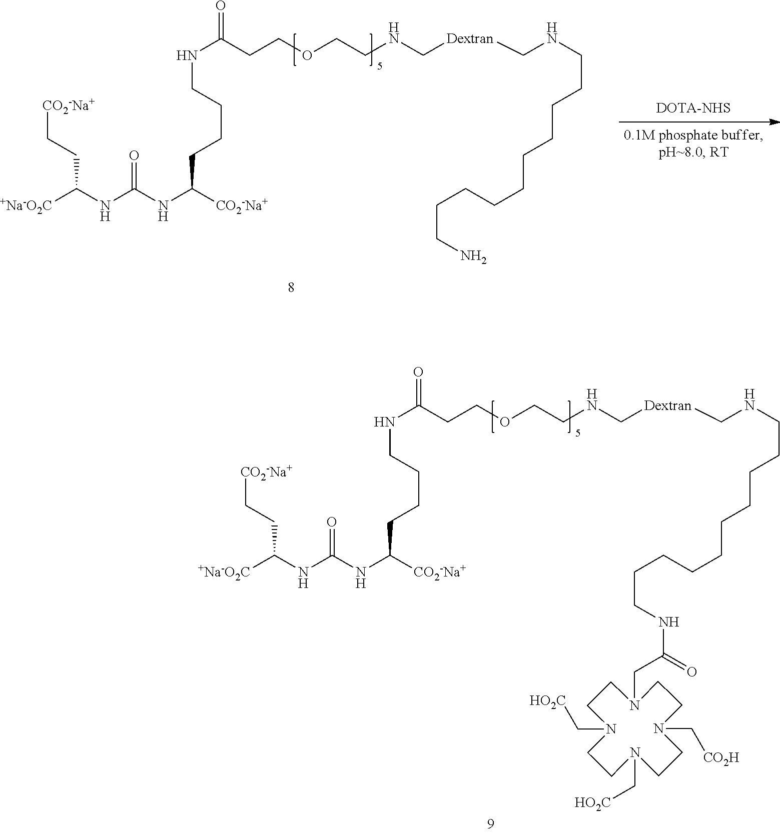

100 mg of nanoparticles lyophilisate (compound 8) was dissolved in 2.0 ml of 0.1M phosphate buffer of pH 8.0. Afterwards, 0.5 ml of DOTA-NHS suspension in ultrapure water, containing 18.5 mg of the chelator, was added. Thus prepared reaction mixture was stirred at room temperature for 90 minutes. The product was purified through dialysis against one hundred-fold volume of 10 mM acetate buffer with pH of 5.0 for 48 hours, with the buffer solution changed six times. Water was removed from thus purified nanoparticles (compound 9) by lyophilisation.

### EXAMPLE 5

Producing Nanoparticles with 1% Aldehyde Group Substitution with the GuL Targeting Agent at 99% Substitution with the DAD Folding Agent

5.1. Oxidation of Dextran to Polyaldehyde Dextran (PAD)

Dextran Oxidation Reaction:

5.00 g of dextran was dissolved in 100 ml ultrapure water. 0.66 g sodium periodate was added. The oxidation reaction was continued overnight in the dark at room temperature. The product was purified through dialysis for 72 hours in one hundred-fold volume of ultrapure water, with the water changed at least twice. The water was removed by evaporation at 40° C.

Determination of Aldehyde Groups in PAD:

100 μl of 0.8 mM hydroxylamine hydrochloride solution, 300 μl of 0.6 M acetate buffer with pH of 5.8 and 20-100 μl of PAD were added to a 2 ml tube, and then ultrapure water (0-80 μl) was added up to total volume 500 μl. The assay was conducted for three different PAD volumes (20, 60 and 100 μl). A control sample was prepared: 100 μl of 0.8 mM hydroxylamine hydrochloride solution, 300 μl of 0.6 M acetate buffer with pH of 5.8 and 100 μl of ultrapure water was added to a tube. The samples were mixed, incubated at 95° C. for 15 minutes, and then incubated at room temperature for 5 minutes. 500 μl of 0.05% TNBS solution was added to every sample. The samples were mixed, incubated in the dark at room temperature for 60 minutes. Once the incubation was completed, the sample absorbance was measured at the wavelength of 500 nm. 300 μl of 0.6 M of acetate buffer with pH 5.8 mixed with 200 μl of ultrapure water was used as a blank sample. Such assays determined a content of aldehyde groups of 480.3 μmol/1 g PAD.

5.2. Reaction of Glu-CO-Lys(OBu^{t})₃NH₂ with the Linker PEGs.

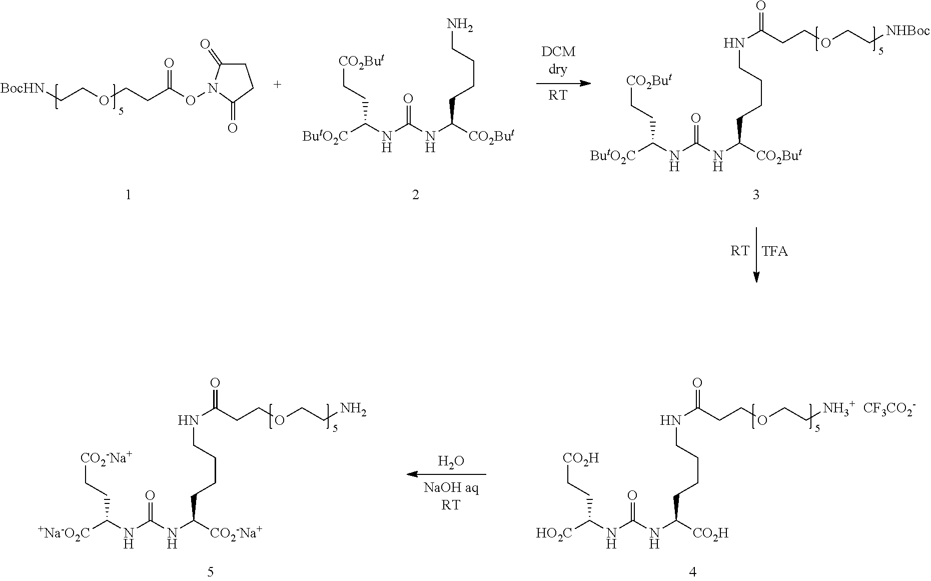

5.20 mg (0.01025 mmol) of the linker (compound 1) was dissolved in 0.25 ml of anhydrous methylene chloride. Afterwards, 5.00 mg (0.01025 mmol) of α,α-urea of glutamic acid and lysine in the form of tert-butyl triesters (compound 2) and 2 μl of DIPEA was added. The reaction was carried out for 24 h at room temperature. After that time, 75 μl of TFA was added, and the mixing was continued over the next 24 h at room temperature. The solvent was evaporated, the oily residue was dissolved in 0.25 ml of ultrapure water, and then alkalised using 5M sodium hydroxide solution to pH>11 against a universal indicator paper. Thus prepared aqueous solution of linker-modified GuL (compound 5) was used for the next stage of the synthesis without purification.

5.3. Formation of Dextran Nanoparticles with Attached Targeting Agent Glu-CO-Lys.

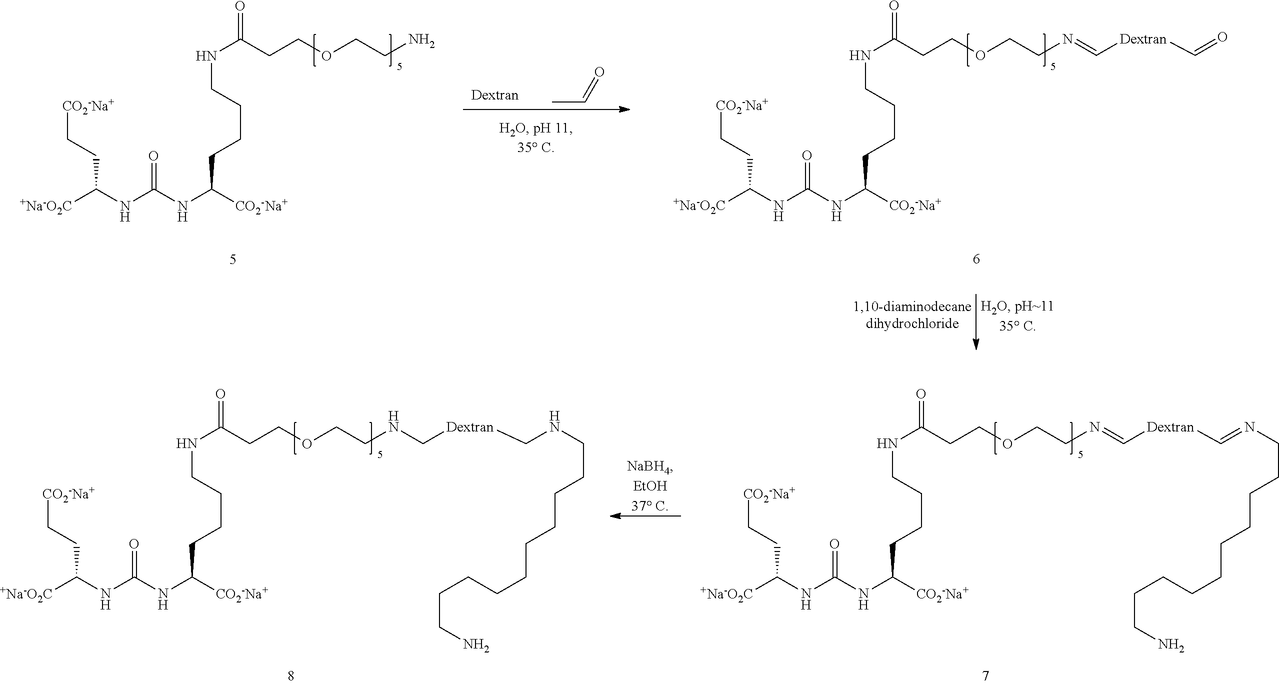

2135 mg of PAD (containing 1025.5 μmol CHO) was dissolved in 21.35 ml of ultrapure water to obtain a 10% (w/v) solution. The aqueous solution of linker-modified GuL (compound 5) was added to that mixture. In thus prepared reaction mixture, 0.5M NaOH solution was used to bring the pH to 11.00, and the mixture was stirred at 30° C. for 60 minutes, resulting in modified polyaldehyde dextran (compound 6). After that time, 12.45 ml of 2% (w/v) ultrapure water solution of 1,10-diaminodecane dihydrochloride was added, and thus obtained reaction mixture was stirred at 30° C. for 10 minutes, with pH controlled and adjusted to 10 every 20 minutes. After the end of the reaction, a 0.5M HCl solution was used to bring the pH to 7.4. Afterwards, 8.84 ml of 1% (w/v) ethanol solution of sodium borohydride was added. The reduction reaction was carried out at 37° C. for 60 minutes. After the end of the reaction, the pH was brought to 7.4 using 0.5M HCl solution. The final product 8 was purified by dialysis in one hundred-fold volume of ultrapure water for 48 h, with water changed six times. Water was removed from thus purified nanoparticles by lyophilisation.

5.4. DOTA Chelator Attachment to Nanoparticles Containing the GuL Targeting Agent

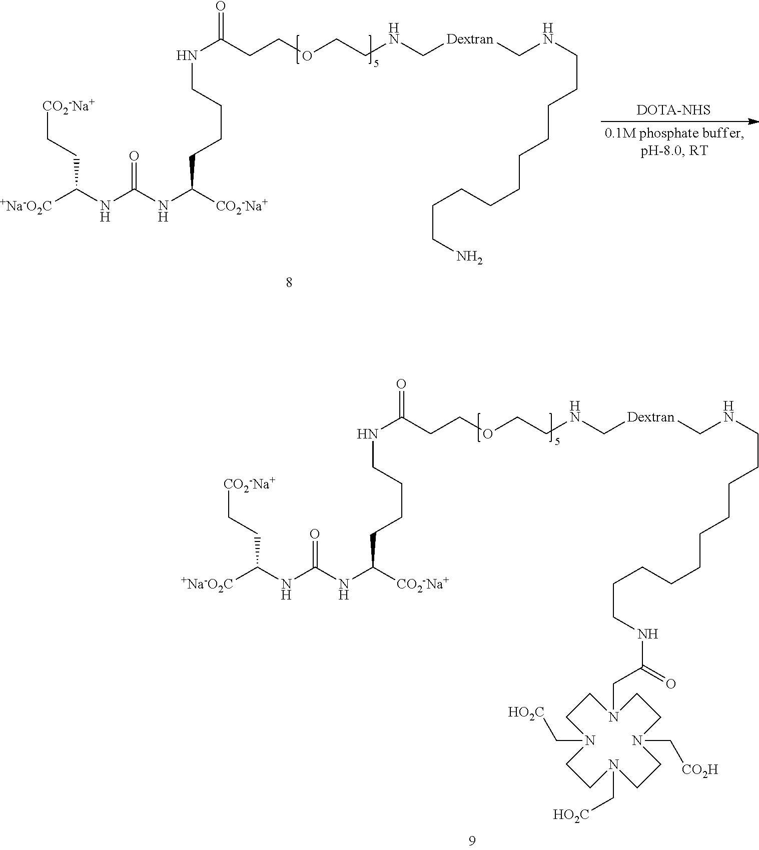

100 mg of nanoparticles lyophilisate (compound 8) was dissolved in 2.0 ml of 0.1M phosphate buffer of pH 8.0. Afterwards, 0.5 ml of DOTA-NHS suspension in ultrapure water, containing 18.5 mg of the chelator, was added. Thus prepared reaction mixture was stirred at room temperature for 90 minutes. The product was purified through dialysis against one hundred-fold volume of 10 mM acetate buffer with pH of 5.0 for 48 hours, with the buffer solution changed six times. Water was removed from thus purified nanoparticles (compound 9) by lyophilisation.

### EXAMPLE 6

Inhibition of PSMA Receptor by Nanoparticles with Attached GuL Targeting Agent

A specificity study of nanoparticles with attached GuL targeting agent embedded on the linker towards the PSMA receptor was performed. An enzymatic in vitro assay was conducted to investigate the decrease in the PSMA activity caused by the blocking of the PSMA active site by the GuL. The study was conducted for the following nanoparticles:
BCS 0277-10% substitution of aldehyde groups with the GuL targeting agent
BCS 0290-30% substitution of aldehyde groups with the GuL targeting agent
BCS 0319-2.5% substitution of aldehyde groups with the GuL targeting agent

for various concentrations of nanoparticles solution used for the analysis, i.e. 16 μg, 4 μg, 1.6 μg, 0.4 μg, 0.16 μg.

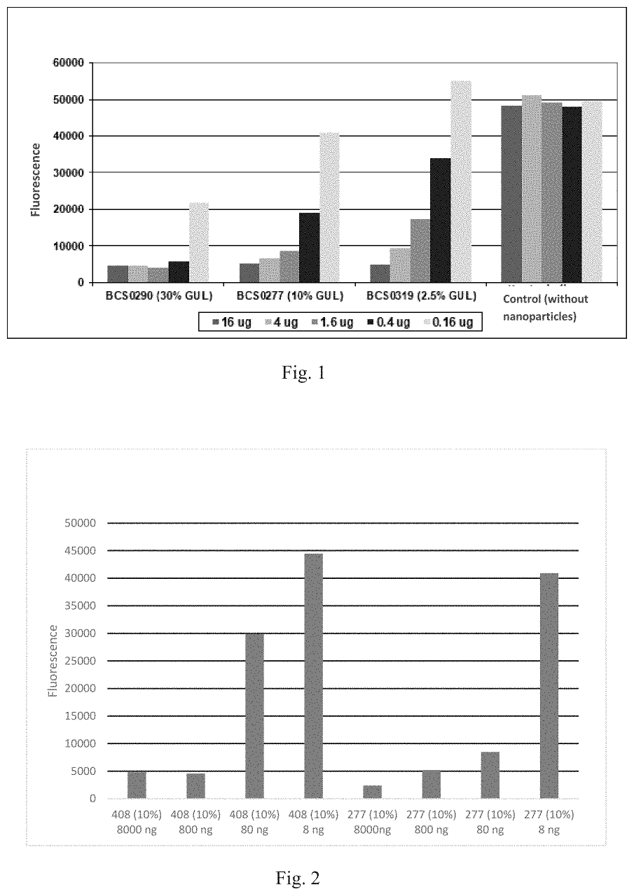

The results are presented in FIG. 1, illustrating the fluorescence drop which reflects the decrease in the enzyme activity. In this way the PSMA inhibition by nanoparticles with an attached GuL targeting agent was established.

The tests have shown that the greater the binding of nanoparticles (GuL content), the lower the fluorescence representing the PSMA enzyme activity. The tendency confirming an increasing amount of bound GuL targeting agent for 30%, 10% as well as 2.5% substitution of the aldehyde groups with the GuL targeting agent was observed. At the same time, the analysis of the results for various values of nanoparticle solution concentrations shows that the presented method permits a quantitative determination of the GuL agent and definition of the minimal nanoparticle concentration required for the inhibition to occur.

The tests are conclusive in proving that, once attached to the nanoparticle structure, the GuL targeting agent placed on the linker has a high affinity for the PSMA receptor present on the surface of prostate cancer cells.

### EXAMPLE 7

Affinity of the Nanoparticles with a GuL Targeting Agent for the PSMA Receptor

The nanoparticles with a GuL targeting agent deposited on the linker were tested for affinity to the PSMA receptor through measurement the degree of its binding on the surface of the LNCaP cells (prostate cancer cell line) exhibiting high overexpression of the PSMA receptor.

The nanoparticles were labelled with radioactive Lutetium and then incubated at 50 μg/ml concentration with LNCaP on a multiwell plate. The nanoparticle binding capacity and internalisation to cells was determined through the measurement of gamma radiation.

The method is characterised by high sensitivity of the measurement.

The results for the following nanoparticles are presented:
BCS 0290-30% substitution of aldehyde groups with the GuL targeting agent
BCS 0318-5% substitution of aldehyde groups with the GuL targeting agent
BCS 0319-2.5% substitution of aldehyde groups with the GuL targeting agent

The results shown in Table 1 suggest that all the tested nanoparticles exhibit high PSMA receptor overexpression. The tests show that nanoparticles with 2.5% to 5% aldehyde group substitution with the GuL targeting agent have a significantly higher level of affinity for the PSMA receptor.

TABLE 1

| | Aldehyde | Binding | | |
|---|---|---|---|---|
| Nano- | group | on the | Internali- | Complete |
| particles | substitution % | surface | sation | binding |
| 290 | 30% | 25.95% | 7.52% | 33.47% |
| 318 | 5% | 29.96% | 2.78% | 32.74% |
| 319 | 2.5% | 46.64% | 10.40% | 57.04% |

### EXAMPLE 8

Testing the Significance of the GuL Targeting Agent Linker for the Specificity of Nanoparticle Binding to the PSMA Receptor

The GuL targeting agent is attached through a linker—a PEG₅ (BocNH-PEG5-NHS) molecule, which is responsible for increasing the access of the targeting agent to the PSMA receptor. Studies have been carried out to confirm the superiority of the GuL-linker molecule on the surface of the nanoparticle over the GuL molecule attached to the nanoparticle without a linker. The results presented in FIG. 2 illustrate PSMA inhibition by nanoparticles with GuL without the linker (408) and with the linker (277) for various quantities of the targeting agent, i.e. 8000 ng, 800 ng, 80 ng and 8 ng.

On the basis of the performed tests, it was found that the decrease in fluorescence reflects the degree of the nanoparticle binding with the GuL targeting agent to the PSMA receptor protein. The results obtained confirm the specificity of the binding of nanoparticles by the targeting agent attached to the linker. They also indicate that the targeting agent with the linker increases the efficiency of the attachment process and the potency of the obtained nanoparticles in relation to the receptor when compared to a targeting agent without a linker.

### Abbreviations

DOTA—1,4,7,10-tetraazacyclododecane-1,4,7,10-tetraacetic acid
DTPA—pentetic acid
NOTA—1,4,7-triazacyclononane-1,4,7-triacetic acid
DOTA-NHS—1,4,7,10-tetraazacyclododecane-1,4,7,10-tetraacetic acid and N-hydroxysuccinimide monoester
DOTA-buthvlamine—1,4,7,10-tetraazacyclododecane-1,4,7-tris(acetic acid)-10-(4-aminobuthyl)acetamide
DOTA-maleimide—1,4,7,10-tetraazacyclododecane-1,4,7-tris-acetic acid-10-maleimidoethylacetamide
DOTA-SCN—2-(4-isothiocyanatobenzyl)-1,4,7,10-tetraazacyclododecane-1,4,7-tris-acetic acid
PET—Positron Emission Tomography
PET/MRI—Positron Emission Tomography and Magnetic Resonance Imaging
NHS—N-hydroxysuccinimide
SulfoNHS—N-hydroxysulfosuccinimide sodium salt
PFP—pentafluorophenol
TFP—2,3,5,6-tetrafluorophenol
STP—2,3,5,6-tetrafluoro-4-hydroxybenzenesulfonic acid sodium salt
SCN—thiocyanate
PAD—polyaldehyde dextran
DAD—diaminodecane
DIPEA—diisopropylethylamine
TFA—trifluoroacetic acid
GuL or Glu-CO-Lys—α,α-urea of glutamic acid and lysine
Glu-CO-Lys(OBu)₃NH₂—α,α-urea of glutamic acid and lysine in the form of tert-butyl triesters

1. A process for preparing polymeric nanoparticles that chelate radioactive isotopes and have their surface modified with specific molecules targeting the PSMA receptor on the surface of cancer cells, characterized in that it comprises the stages in which:
a) a dextran chain is oxidized to polyaldehyde by means of periodate,
b) a targeting agent that is α,α-urea of glutamic acid and lysine, the targeting agent modified by a linker molecule is attached to free aldehyde groups present in the dextran chain,
c) a folding agent in the form of hydrophobic diamine or polyamine is attached, with one or two amino groups of the folding agent attaching to aldehyde groups,
d) the resulting imine bonds are reduced to amine bonds,
e) to the free amino group of the attached folding agent, a chelator molecule is attached via an amide bond,
f) the resulting mixture is purified,
g) the nanoparticle fraction is subjected to lyophilization.

2. The process according to claim 1, wherein the mixture from stage f) is purified by dialysis.

3. The process according to claim 1, wherein the cells on which the PSMA receptor is present are prostate cancer cells and metastatic prostate cancer cells.

4. The process according to claim 1, wherein the cells on which the PSMA receptor is present are breast, lung, colon and pancreatic cancer cells.

5. The process according to claim 1, wherein the substitution of the aldehyde groups with the targeting agent is from 1 to 50%.

6. The process according to claim 5, wherein the substitution of the aldehyde groups with the targeting agent is from 2.5 to 5%.

7. The process according to claim 1, wherein the chelators are derivatives of DOTA, DTPA and/or NOTA.

8. (canceled)

9. The process according to claim 1, wherein the linker is 2,5-dioxopyrrolidin-1-yl 2,2-dimethyl-4-oxo-3,8,11,14,17,20-hexaoxa-5-azatricos-23-ate (PEG₅).

10. The process according to claim 1, wherein the folding agent are lipophilic diamines selected from the group consisting of dodecylamines, diaminooctanes, diaminodecanes (DAD), polyether diamines, polypropylene diamines and block copolymer diamines.

11. The process according to claim 1, wherein the obtained nanoparticles are radiochemically labelled with such isotopes in which the breakdown pathway involves beta plus decay, beta minus decay, gamma emitter decay.

12. Polymeric nanoparticles chelating radioactive isotopes, with a surface modified by specific molecules targeting the PSMA receptor as obtained according to the process of claim 1, for use in diagnostics and therapy.

13. Polymeric nanoparticles chelating radioactive isotopes according to claim 12 for use in Positron Emission Tomography PET and PET/MRI diagnostics.

14. Polymeric nanoparticles chelating radioactive isotopes according to claim 12 for use in focal brachytherapy.

15. Polymeric nanoparticles chelating radioactive isotopes prepared according to the process of claim 1 for use in the therapy and diagnostics of prostate cancer and metastatic cancer as well as other cancers with affected cells to which the nanoparticles show the affinity.
